(19) United States
(12) Patent Application Publication
(10) Pub. No.: US 2020/0015440 A1
(43) Pub. Date: Jan. 16, 2020

(54) PRODUCTION OF CANNABIS PLANTS AND SEEDS USING A TARGETED ALLELE

(71) Applicant: JACK HEMPICINE LLC, Albany, OR (US)

(72) Inventors: Seth S. Crawford, Corvallis, OR (US); Eric B. Crawford, Eugene, OR (US)

(21) Appl. No.: 16/510,032

(22) Filed: Jul. 12, 2019

Related U.S. Application Data
(60) Provisional application No. 62/697,365, filed on Jul. 12, 2018.

(51) Int. Cl. A01H 1/02; A01H 6/28; A01H 5/12

(52) U.S. Cl. CPC A01H 1/02; A01H 5/12; A01H 6/28

(57) ABSTRACT

A method for developing seeds for CBGa dominant Cannabis plants includes self-pollinating a female Cannabis plant having at least one defective CBDa synthase allele to produce seeds, and collecting the seeds produced through self-pollinating. A method for developing seeds for CBGa dominant Cannabis plants includes crossbreeding a Cannabis plant having at least one defective CBDa synthase allele with a Cannabis plant having at least one of an active THCa or CBDa synthase allele to produce a CBGa dominant plant, and collecting the seeds produced through the crossbreeding. A Cannabis seed, developed from one of self-pollinating a Cannabis plant having a defective CBDa synthase allele or crossbreeding the Cannabis plant with another Cannabis plant that has one of either an active THCa or an active CBDa synthase allele resulting in a CBGa dominant seed.

### CROSS REFERENCE TO RELATED APPLICATION

This application claims priority to and the benefit of U.S. Provisional Application No. 62/697,365 filed Jul. 12, 2018, which is incorporated herein by reference in its entirety

### FIELD OF THE INVENTION

This disclosure is directed to the identification and use of particular CBDa synthase alleles, more particularly to using these alleles to produce Cannabis plants having very high ratios of CBGa to CBDa and/or THCa.

### BACKGROUND

Cannabis is a genus of plants useful in the industrial or artisanal production of oil, fiber, food, fragrance, and medicine. The various parts of Cannabis plants may be used in a near infinite number of products, such as fiber, oils, and medicines, for example.

THC makes up the psychoactive portion of Cannabis and leads to the ‘high’ associated with the use of Cannabis. Federal regulations consider any Cannabis plant having any level of THC to be a Schedule 1 drug, those that are not to be manufactured or sold for any reason. Many states that have legalized marijuana consider plants and their products to be “THC-free” if they have less than 0.5% THC. However, federal regulations still apply, typically to those products having 0.3% or higher THC. This issue becomes compounded when the plants have their phytocannabinoids removed and concentrated, as that process can raise the level of THC in the resulting product.

Therefore, Cannabis plants having a complete, or nearly complete, inability to produce THC gives rise to plants that are 100% legal as industrial hemp in any country and would remove them from federal regulation in the US.

### BRIEF DESCRIPTION OF THE DRAWINGS

FIG. 1 shows a graph of chemotype segregation for an F2 generation from a type III population.

FIG. 2 shows a distribution of type III and IV plants from an F2 population.

FIG. 3 shows an exploded view of the IV distribution of FIG. 2.

### DETAILED DESCRIPTION

Plant species in the genus, Cannabis, are annual plants that are wind-pollinated to produce seeds that germinate the following year. Cannabis plants are dicotyledons that bear fruit in the form of achenes, which consist of one seed protected by two cotyledons or bracts (i.e., embryonic leaves) as well as energy rich nutritional proteins with essential amino acids.

Cannabis plant species are dioecious, meaning that staminate plants with a male sex chromosome, XY, have male flowers containing microgametophytes within the pollen, and pistillate plants with only female sex chromosomes, XX, have female flowers containing megagametophytes within the ovules. Hermaphroditic plants and flowers are also possible in monoecious phenotypes. Morphological differences for visually distinguishing between male and female plants develop during the reproductive stage.

The diurnal light cycle and/or exposure to low levels of carbon monoxide may change the gender expression of a plant. Female plants may be treated with silver thiosulfate (STS), causing them to produce pollen sacs instead of female flowers.

The life cycle of Cannabis plants includes germination/emergence, vegetative growth, reproductive stages, in which flowers and seeds are formed, and finally, senescence. The time for maturation may vary from about 2 to 10 months, but naturally, the time from seed to harvest is about 8 months. However, artificial indoor growing operations can speed the life cycle of Cannabis plants to just 90 days by boosting light exposure and tightly controlling the timing of the required photoperiods.

Cannabis seeds mostly lack dormancy mechanisms and germinate without requiring any pre-treating or winterizing. Weights range from about 2 to 70 grams per 1,000 seeds. When placed in viable growth conditions, Cannabis seeds germinate in about 1-19 days.

The stages of vegetative growth include time as a juvenile or basic vegetative phase and a photosensitive phase, lasting until the development of flowers. Vegetative growth may last for about 2-20 weeks, during which growth increases in response to temperature and increasing light exposure, and plants may be grown to their desired size. After the juvenile stage of about 1-8 weeks, plants require at least 12 hours of light before flowering may begin about 1-12 weeks later. Exposing the plants in the photosensitive phase to a critical photoperiod, about 14-16 hours of light, begins flower development. In general, about 18-20 hours of light per day during the vegetative growth stage has been shown to produce the highest yields for some Cannabis plant varieties. Interrupting the continuity of just one night or darkness period during the photosensitive phase of the plant can delay or disrupt flower maturation. Exposure to just one or two periods of short days, or long nights, may induce flowering. In day-neutral, or autoflowering, plants, entering the flowering stage may be irreversible.

Typically, sun-grown Cannabis plants flower between July and September, depending on the latitude. The flowering stage may range from about 6 to 16 weeks, depending on the genetics and environment. After the initially developed flowers that have been pollinated produce their fruit and seeds, pistillate plants may continue to produce additional flowers while staminate plants die. Colder weather eventually kills pistillate plants unless they are grown indoors or artificially induced into a vegetative state.

After being grown, Cannabis plants may be harvested at full flowering or at the end of flowering for their fiber, seeds, or cannabinoids. Indicators that plant flowers are ready for harvest may include stigmas changing color or disappearing.

Cannabis plants uniquely contain C₂₁ or C₂₂ terpenophenolic chemical compounds known as cannabinoids—specifically, phytocannabinoids that naturally occur within the plant itself. Many Cannabis crops are harvested specifically to collect these cannabinoids for various downstream uses, so often plant varieties are bred to maximize their total cannabinoid yield. The phytocannabinoids within a Cannabis plant are secondary metabolites synthesized within glandular trichome cells and may include cannabigerolic acid (CBGa), which can be converted into cannabichromenic acid (CBCa), cannabidiolic acid (CBDa), and/or tetrahydrocannabinolic acid (THCa) depending on the type of enzymes present in the plant according to its genetics. Specifically, the oxidoreduction and cyclization of CBGa catalyzed by THCa and CBDa synthases provides the synthesis of THCa and CBDa. The phytocannabinoid content resulting from the plant's genetics allow for classification of Cannabis plant types by discrete chemical phenotype or chemotype, as shown in Table A below.

TABLE A
CANNABIS PLANT TYPE CATEGORIZATION

| Cannabis | |
|---|---|
| Chemotype | Phytocannabinoid Content Description |
| Type I | THCA dominant |
| Type II | Substantially equal parts CBDA and THCA |
| Type III | CBDA dominant |
| Type IV | CBGA dominant |
| Type V | Cannabinoid free |
| | (i.e., containing terpenes but no cannabinoids) |

The overall THCa/CBDa ratio is thought to be genetically predetermined and thus, does not vary significantly throughout the life of the plant. The THCa and CBDa synthases were first believed to be codominant alleles at a single locus. However, as discovered by Weiblen, et al. they are actually two separate genes located 8 centimorgans apart on the same chromosome (Weiblen, et al. “Gene Duplication and Divergence Affecting Drug Content in Cannabis Sativa.” New Phytologist, 2015).

Additionally, the expression of the CBGa pure or dominant chemotype for Type IV plants may have resulted from self-fertilization or inbreeding within monoecious or hermaphroditic plants creating a fixed, mutated B₀ allele. The expression of the cannabinoid-free chemotype for Type V plants may be due to null genotypes at an A locus. Further chemotype expressions are possible, such as CBCA synthase encoding with the B_{C} allele, for example. The frequency of the THCa synthase allele (B_{T}) and plants with propyl sidechain cannabinoids have been found to be higher within Cannabis indica varieties than in varieties of Cannabis sativa and Cannabis ruderalis. Often, however, the different chemotypes or varieties of Cannabis plants are crossbred, leading to interesting new traits but increasing the heterozygosity of the resulting progenies. The resulting plants grown from the seeds created from crossbreeding two parent plants are referred to as F₁ progeny plants. F₁ progenies often have reduced homozygosity, causing instability in their expressed traits.

The homozygosity, genetic variation, of Cannabis plants may be measured in terms of the amount of polymorphism observed in scoring randomly amplified polymorphic DNA markers and/or performing amplified fragment length polymorphism analyses. Moreover, the bulk segregant analysis strategy for finding molecular markers may be used when F2 progenies, from interbreeding F₁ individuals, exhibit clear cut segregation.

Inbreeding plants involves some form of self-crossing or asexual propagation, such as by cloning by self-pollination or clipping, which may reduce the amount of heterozygosity within the genetics. For example, experiments have shown that doubly inbred plants, such as S₂ progenies, exhibit less genetic variation as compared to non-inbred plants.

If self-crossed plants, S₁ or S₂ progenies, are crossed such that the resulting plants, F₁ progenies, segregate into distinct phenotypes, it indicates that the self-crossed parent plants were still heterozygous at the relevant loci. In general, the CBDa content within heterozygous F₁ progenies of crossed pure homozygous chemotypes, Types I and III, is higher than that of Type III parent plants derived from fiber strains with lower inflorescence density and total cannabinoid content. Further, deviations from a strictly even dispersal within the tripartite cannabinoid ratio distribution model among F2 progenies may indicate a natural preference away from the Type III chemotype due to a recessive and unfavorable factor that corresponds to the B_{D} allele, evidenced in the significantly reduced fertility of pure CBDa plants expressed during embryogenesis.

Industrial Cannabis plants, those with less than 0.3% THCa, have an innumerable variety of uses, including hemp products and oil.

The methods discussed below may include back-crossing or self-crossing varieties to produce purer species with reduced heterozygosity from hybridized strains. Reducing heterozygosity may involve inbreeding female plants until a fixed homozygous phenotype is achieved. For example, Cannabis sativa plants may be bred among themselves through interbreeding or self-breeding until producing a set of plants that are each sufficiently homozygous. Additionally, or alternatively, Cannabis ruderalis plants may be similarly bred among themselves until a sufficient level of homozygosity is reached. Reducing heterozygosity may require several generations of breeding depending on the level of homozygosity or purity desired. The process of reducing homozygosity may result in the creation of an inbred line that produces plants with minimized differences between each other. As described above, homozygosity or homology may be measured through genetic testing and/or other method of morphological, varietal, biotypical, or phenotypical identification. Embodiments here may involve homogenizing the resulting plants for at least 3 generations.

Self-fertilization, self-pollination, or self-crossing of plants may be performed by hand-pollinating female flowers with pollen from induced male flowers on the same plant. The male flowers may be induced by the application of an aqueous solution of silver nitrate (AgNO₃) to the growing shoot tip of the female plant, in accordance with the method disclosed in Ram et al., “Induction of Fertile Male Flowers in Genetically Female Cannabis sativa Plants by Silver Nitrate and Silver Thiosulphate Anionic Complex”, Theor. Appl. Genet. 62, 369-375 (1982), which is herein incorporated by reference. The seeds bore from the self-pollination may produce only pistillate female plants due to their genetics containing only female sex chromosomes.

Other methods for self-pollination, whether through inducing fertile male flowers on pistillate plants, such as by using colloidal silver, gibberellic acid, or Rodelization, feminization of staminate plants, such as by using ethephon, or alternative means such as irradiation, streptovaricin treatment, are also possible. The embodiments here use these techniques as set out below to develop CBGa dominant plants.

In addition to cannabinoids, Cannabis plants also include aromatic secondary metabolites, such as flavonoids and terpenoids or terpenes. Such terpenoids (e.g., mono-, di-, and sesquiterpene oils) or flavonoids may include α-bisabolol, borneol, isoborneol, menthol, nerol, camphene, camphor, Δ³-carene, α-cedrene, β-eudesmol, eudesmol, fenchol, geraniol, β-myrcene, myrcene, α-terpinene, α-terpineol, α-terpinolene, terpinolene, α-phelladerene, α-pinene, β-pinene, pinene, sabinene, α-humulene, humulene, β-caryophyllene, caryophyllene oxide, trans-caryophyllene, cis-ocimene, trans-ocimene, geranyl diphosphate, farnesol, leucosceptrine, squalene, limonene, phytol, guaiol, and linalool, for example.

It has been found that each Cannabis biotype (e.g., Cannabis sativa, Cannabis indica, Cannabis ruderalis) has commonalities among the terpene profiles of its strains. For example, Cannabis sativa strains may be called Diesel due to the higher levels of terpenes such as humulene and/or β-caryophyllene. The interaction of specific terpenes with the receptors in mammalian brains and bodies may affect the binding of both endocannabinoids and phytocannabinoids. Thus, selection for a terpene profile within a plant may be application specific.

As mentioned above, Cannabis plants of the F₁ generation may be further selected for breeding based on their organoleptic appeal due to resin, cannabinoid, and/or terpene levels. Such categories of aromatic selection may include, but are not limited to, berry, citrus, pine, lemon, and/or diesel.

The F₁ generation of Cannabis plants may further be selected for seed and/or fiber yield and/or quality, depending on the industrial application.

There exists CBDa synthase alleles that appear to be full-length, meaning that they are an active allele but it is defective. Defective, nonfunctional alleles are typically truncated. These CBDa synthase alleles may contain a frame shift mutation that renders them nearly completely incapable of producing any CBDa or THCa. This may allow targeted use of these alleles to create varieties that have nearly or no THCa and therefore fall below the federal limit of 0.3% for hemp. This allows production of seeds and plants that are 100% legal as industrial hemp in any country, even at full maturity in the field. In addition, plants with the levels of purity of CBGa:THCa of 300:1+ can have their phytocannabinoids removed and concentrated and still remain below the federal limit. These full-length but inactive alleles will be referred to as ‘defective’ alleles and ‘normal’ alleles that are both full-length and active will be referred to as ‘active’ alleles.

It has been determined that plants having one copy of this defective allele have a small bioaccumulation of CBGa in the range of 0.5% to 2.5% as a secondary cannabinoid in type I and type III plants and as a tertiary cannabinoid in type II plants. Two copies of the allele create type IV “pure” CBGa plants with CBGa:THCa ratios of approximately 100:1. The embodiments include an even more pure plant, having ratios in the range of 300:1, for both CBGa to THCa and CBGa to CBDa, with further inbreeding. The presence of a number of heterozygous alleles has been demonstrated by several independent research groups via genome analysis.

### EXAMPLE

Under the industrial hemp research legalization within Section 7606 of the Agricultural Act of 2014, the following experiments were conducted as part of Oregon's agricultural pilot program for the growth, cultivation, and marketing of industrial hemp.

In a first example, an “ultra-pure,” individual plant, having two copies of the defective CBDa allele, was crossed with a standard type III, CBD dominant plant. This resulted in progeny that were still CBD dominant, having ratios of CBD:THC in the range of 27:1-33:1. Typical ratios in type III plants are in the range from 10:1-40:1.

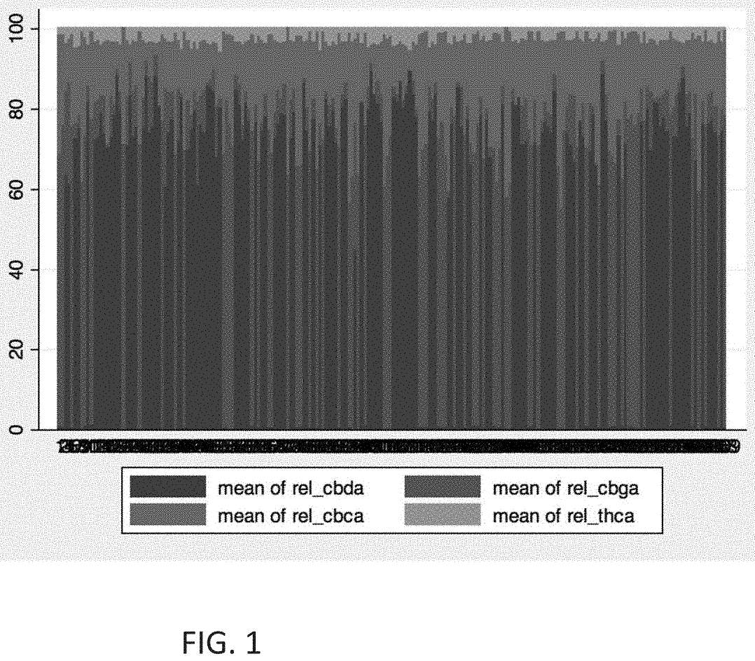

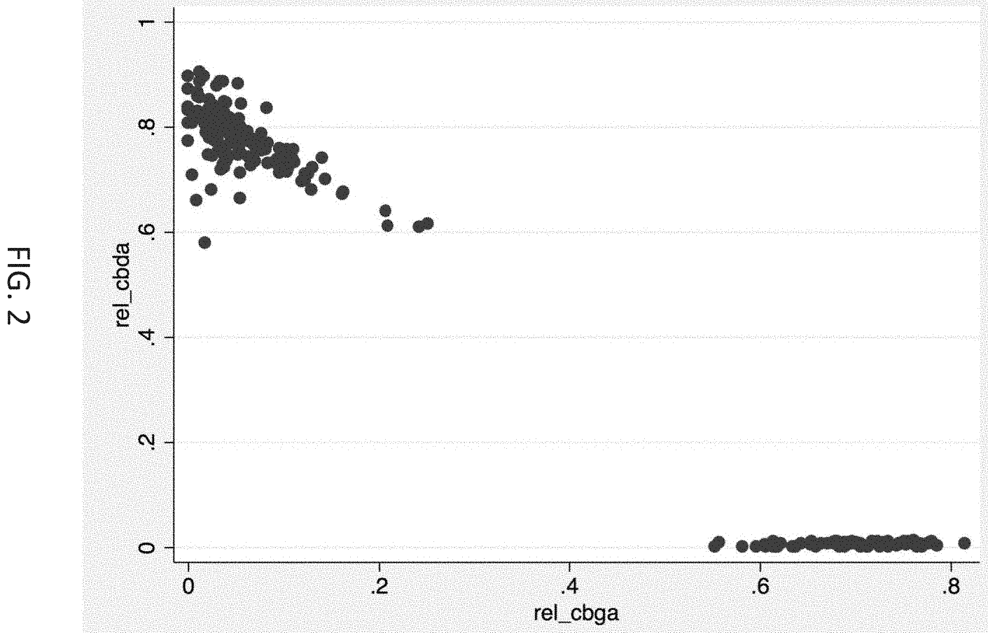

In this example, a type IV progeny, having two copies of the defective CBDa allele, of a type III plant that naturally contained more CBGa than typically found in type III plants was created. Its characteristics are found in the Appendix. This was then crossed with another type III line referred to as ERB. The resulting progeny, F1, were all type III CBDa dominant, but contained elevated levels of CBGa as a secondary compound. Forty individuals from the F1 population were open pollinated to produce the F2 generation, in which major chemotype segregation occurred as shown in FIG. 1 and FIG. 2. FIG. 2 shows clusters of the type III plants in the upper left corner, and type IV plants in the lower right corner.

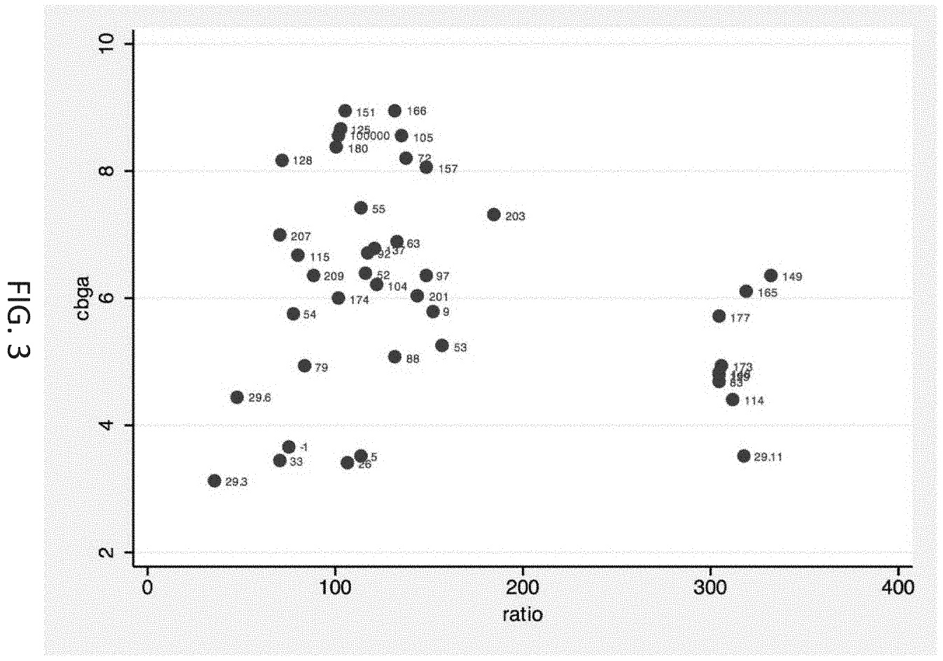

The type IV individuals from this F2 population were identified and selected. These selected individuals then underwent flowering and HPLC chemical analysis. There was an additional divergence between the roughly 100:1 individuals and the rarer 300:1 individuals, as shown in FIG. 3 as a more detailed view of the type VI cluster from FIG. 2.

Essentially, the examples involve developing seeds for CBGa dominant Cannabis plants by self-pollinating a female Cannabis plant having at least one defective CBDa synthase allele to produce seeds, and then collecting the seeds produced through self-pollinating. They also involve developing seeds for CBGa dominant Cannabis plants by crossbreeding a Cannabis plant having at least one defective CBDa synthase allele with a Cannabis plant having an active THCa synthase allele to produce a CBGa dominant plant, and collecting the seeds produced through the crossbreeding. Finally, the embodiments include Cannabis seed, developed from one of self-pollinating a Cannabis plant having a defective CBDa synthase allele or crossbreeding the Cannabis plant with another Cannabis plant that has an active THCa synthase allele to produce a CBGa dominant seed.

The previously described versions of the disclosed subject matter have many advantages that were either described or would be apparent to a person of ordinary skill. Even so, these advantages or features are not required in all versions of the disclosed apparatus, systems, or methods.

Additionally, this written description makes reference to particular features. It is to be understood that the disclosure in this specification includes all possible combinations of those particular features. For example, where a particular feature is disclosed in the context of a particular aspect, that feature can also be used, to the extent possible, in the context of other aspects.

Also, when reference is made in this application to a method having two or more defined steps or operations, the defined steps or operations can be carried out in any order or simultaneously, unless the context excludes those possibilities.

Although specific aspects of the invention have been illustrated and described for purposes of illustration, it will be understood that various modifications may be made without departing from the spirit and scope of the invention. Accordingly, the invention should not be limited except as by the appended claims.

The aspects of the present disclosure are susceptible to various modifications and alternative forms. Specific aspects have been shown by way of example in the drawings and are described in detail herein. However, it should be noted that the examples disclosed herein are presented for the purposes of clarity of discussion and are not intended to limit the scope of the general concepts disclosed to the specific aspects described herein unless expressly limited. As such, the present disclosure is intended to cover all modifications, equivalents, and alternatives of the described aspects in light of the attached drawings and claims.

References in the specification to aspect, example, etc., indicate that the described item may include a particular feature, structure, or characteristic. However, every disclosed aspect may or may not necessarily include that particular feature, structure, or characteristic. Moreover, such phrases are not necessarily referring to the same aspect unless specifically noted. Further, when a particular feature, structure, or characteristic is described in connection with a particular aspect, such feature, structure, or characteristic can be employed in connection with another disclosed aspect whether or not such feature is explicitly described in conjunction with such other disclosed aspect.

We claim:

1. A method for developing seeds for CBGa dominant Cannabis plants, comprising:
self-pollinating a female Cannabis plant having at least one defective CBDa synthase allele to produce seeds; and
collecting the seeds produced through self-pollinating.

2. The method of claim 1, wherein the female Cannabis plant is Cannabis sativa.

3. The method of claim 1, wherein the female Cannabis plant is Cannabis ruderalis.

4. The method of claim 1, wherein self-pollinating includes:
inducing staminate flowers on a pistillate plant; and
pollinating pistillate flowers on the pistillate plant with pollen from the induced staminate flowers.

5. The method of claim 1, further comprising homogenizing the initial Cannabis plant for at least 3 generations.

6. A method for developing seeds for CBGa dominant Cannabis plants, comprising:
crossbreeding a Cannabis plant having at least one defective CBDa synthase allele with a Cannabis plant having at least one of an active THCa or CBDa synthase allele to produce a CBGa dominant plant; and
collecting the seeds produced through the crossbreeding

7. The method of claim 6, wherein the Cannabis plant having the defective CBDa synthase allele is Cannabis sativa.

8. The method of claim 6, wherein the Cannabis plant having the defective CBDa synthase allele is Cannabis ruderalis.

9. The method of claim 6, further comprising homogenizing the initial Cannabis plant for at least 3 generations.

10. The method of claim 6, wherein the Cannabis plant with the defective CBDa synthase allele is a type IV plant.

11. The method of claim 6, further comprising:
self-pollinating progeny from the cross-breeding;
identifying and selecting individual progeny that are type IV; and
causing the individual progeny to flower, prior to collecting the seeds.

12. A Cannabis seed, developed from one of self-pollinating a Cannabis plant having a defective CBDa synthase allele or crossbreeding the Cannabis plant with another Cannabis plant that has one of either an active THCa or an active CBDa synthase allele, comprising:
a CBGa dominant seed.

14. The Cannabis seed of claim 12, wherein the seed also has a ratio of at least 100:1 of CBGa to THCa.

15. The Cannabis seed of claim 12, wherein the ratio is at least 300:1 of CBGa to THCa.

16. The Cannabis seed of claim 15, wherein the seed also has a ratio of at least 300:1 of CBGa to CBDa.
